(19) United States
(12) Patent Application Publication
(10) Pub. No.: US 2006/0161074 A1
(43) Pub. Date: Jul. 20, 2006

(54) Temperature sensing device

(76) Inventor: Chi-Hong Liao, Taipei County (TW)

Correspondence Address: MORLAND C FISCHER, 2030 MAIN ST, SUITE 1050, IRVINE, CA, 92614, US

(21) Appl. No.: 11/040,046

(22) Filed: Jan. 20, 2005

(51) Int. Cl. A61B 5/00

(52) U.S. Cl. 600/549

(57) ABSTRACT

A temperature sensing device is proposed. The device includes a body and at least a temperature sensing member separated from the body by a predetermined distance. The body includes a displaying unit and a first interface unit, and the temperature sensing member is connected to the first interface unit of the body via a second interface unit. The temperature sensing member is set on a test subject using a detachable fixing unit and a measuring unit is used to measure the body temperature of the test subject. The measurement is received and processed by a signal processing unit, and subsequently transmitted to and displayed in the displaying unit. The signal processing unit can also be provided in the body or the temperature sensing member for converting and processing a temperature signal.

### FIELD OF THE INVENTION

The present invention relates to a temperature sensing device, and more particularly, to a temperature sensing device which is able to detect body temperature changes when in contact with a test subject.

### BACKGROUND OF THE INVENTION

Commonly marketed temperature sensing devices such as mercury thermometers, ear thermometers, electronic thermometers, infrared thermometers, and temperature measuring cards are employed to measure body temperature of a test subject using a contact or non-contact method, such that the devices can determine whether the body temperature of the test subject falls within a normal temperature range.

In general, temperature sensing devices such as the foregoing mercury thermometer, ear thermometer, or electronic thermometer are usually used to measure the body temperature of the test subject when the test subject is sick or receives a health check-up or treatment in a clinic or hospital. In other words, except for a person with a special need or an in-patient, not many people measure their own body temperature on a frequent basis. However, as fever is a key symptom to define an infectious period of a communicable disease such as severe acute respiratory syndrome (SARS), body temperature changes have become important indicia for preventing the spread of such diseases.

Therefore, regardless of whether employed by government organizations, private enterprises, or in the home, measuring body temperature is now considered as a basic step to both early-detect disease and prevent the spread of contagious disease. Apart from checking body temperature at the entrances of buildings, offices, and factories, companies increasingly measure body temperature of employees during the work day. Parents and teachers also measure children's body temperatures, and even isolated patients' body temperatures also need to be monitored to monitor their conditions. With the concern that communicable diseases are on the rise, such trends are likely to continue. Thus, there is an increasing need for temperature sensing devices that measure body temperature in a reliable and convenient fashion.

The foregoing temperature sensing devices can be employed to measure the body temperature of the test subject in a contact or non-contact method. However, such temperature sensing devices often have to be held by one person to measure the body temperatures of a number of test subjects. Therefore, the process of measuring the body temperature is very time-consuming and causes inconvenience as well as influences one's working efficiency.

In light of the drawback of the foregoing temperature sensing devices, a portable temperature sensing device has been disclosed, such that the test subject is able to immediately measure the body temperature and record and/or report his present body temperature. However, as such portable temperature sensing device does not directly contact with the test subject, the temperature measurements might be easily affected by environmental temperature to therefore lead to inaccuracy in temperature measurement, just like what has commonly happened when using other non-contact type temperature sensing devices.

Furthermore, regardless of whether using the foregoing temperature sensing devices or the portable temperature sensing device, body temperature change can only be obtained by comparing body temperatures measured at different time points. In other words, temperature-sensing devices known in the prior-arts are not able to provide the function of monitoring body temperature changes because prior body temperature measurements are not stored and available for comparison with current measurements, particularly in situations where one instrument is utilized with multiple test subjects.

The problem to be solved here, therefore, is to provide a temperature sensing device which eliminates the prior-art drawbacks of requiring excessive operator time and measuring inaccuracy in a body temperature measurement while easily monitoring body temperature changes.

### SUMMARY OF THE INVENTION

The primary objective of the present invention is to provide a temperature sensing device which is capable of monitoring body temperature changes.

Another objective of the present invention is to provide a temperature sensing device which is able to measure body temperature at a distance.

Still another objective of the present invention is to provide a temperature sensing device which is able to improve the accuracy of body temperature measurements.

A further objective of the present invention is to provide a temperature sensing device which is able to measure body temperatures according to different monitoring modes.

In accordance with the above and other objectives, the present invention proposes a temperature sensing device, comprising a body and a detachable temperature sensing member freely provided in the body. The body comprises a displaying unit and a first interface unit. The temperature sensing member has a measuring unit and a fixing unit and is separated from the body by a predetermined distance and connected to the body by a second interface unit via the first interface unit. Thus, the temperature sensing member can be set on a test subject using the fixing unit and body temperature of the test subject can be measured using the measuring unit. A measurement is then received, converted, and processed by a signal processing unit, such that the converted and processed result of measurement is subsequently transmitted to and displayed on the displaying unit. The signal processing unit can be preferably provided in the body and/or the temperature sensing member to convert and process the measurements.

The present invention is characterized in that the body and the temperature sensing member can be connected to or separated from each other. When the body and the temperature sensing member are connected to each other, body temperature of the test subject can be directly measured to improve on the accuracy of the body temperature measurement. When the body and the temperature sensing member are separated from each other, the temperature sensing member can be set on the test subject and utilized in a single to single (one body to one temperature sensing member), single to multiple (one body to multiple temperature sensing members) and/or multiple to multiple (multiple bodies to multiple temperature sensing members) manner, as desired. Therefore, body temperature of one test subject or a number of test subjects can be simultaneously measured while also providing for the monitoring of body temperature changes.

Moreover, as the temperature sensing device is designed to monitor the body temperature change of one or more subjects, accurate body temperature measurements can be obtained without consuming a large amount of time in testing individuals and without adversely affecting one's working efficiency while performing distant body temperature measurements according to different monitoring modes.

The present invention is described in the following with specific embodiments, so that one skilled in the pertinent art can easily understand other advantages and effects of the present invention from the disclosure of the invention. The present invention may also be implemented and applied according to other embodiments, and the details may be modified based on different views and applications without departing from the spirit of the invention.

### BRIEF DESCRIPTION OF THE DRAWINGS

The present invention can be more fully understood by reading the following detailed description of the preferred embodiments, with reference made to the accompanying drawings, wherein:

FIG. 1 is a block diagram showing a temperature sensing device according to the first embodiment of the present invention;

FIG. 2 is a block diagram showing a body according to the first embodiment of the present invention;

FIG. 3 is a block diagram showing a temperature sensing member according to the first embodiment of the present invention;

FIG. 4 is a block diagram showing a temperature sensing device according to the second embodiment of the present invention;

FIG. 5 is a block diagram showing a body according to the second embodiment of the present invention; and

FIG. 6 is a block diagram showing a temperature sensing member according to the second embodiment of the present invention.

### DETAILED DESCRIPTION OF THE EMBODIMENTS

The following embodiment only serves to provide further description for the present invention and is not intended to limit the scope of the invention.

The First Embodiment

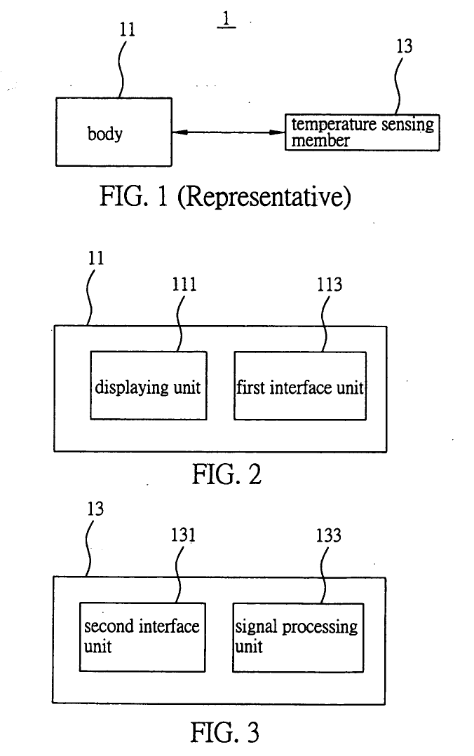

FIG. 1 to FIG. 3 are drawn according to the first embodiment of the present invention. A temperature sensing device 1 comprises a body 11 and a detachable temperature sensing member 13 freely provided in the body 11. The drawings are simplified diagrams and only elements relevant to the present invention are demonstrated. The actual shape and dimension ratios are not further described as they are not technical features of the present invention and can be modified depending on practical requirements.

Referring to FIG. 2, the body 11 comprises a displaying unit 111 and a first interface unit 113. The displaying unit 111 can be a screen for displaying the result of a body temperature measurement. The displayed result of body temperature measurement can be a value (such as 36° C. which can be an initial temperature parameter value and/or a temperature parameter value obtained after re-measurement, as subsequently described), a chart (such as a temperature curve), or other appropriate display methods. In the present embodiment, the displaying unit 111 serves to display the result of body temperature measurement. In addition, the displaying unit 111 can also display data such as an instantaneous value, a maximal value, and a minimal value of body temperature, as well as the time of measuring body temperature. Furthermore, the displaying unit 111 can also display the power supply status of the body 11. The element for supplying power for the body 11 can be any of the power sources (such as a battery) known in the prior-art.

The first interface unit 113 can be used as wired or wireless signal connection between the body 11 and the temperature sensing member 13, such that signals sent out by the temperature sensing member 13 are received and subsequently transmitted to the displaying unit 111 to be displayed. The first interface unit 113 can be a wired or wireless transmission interface selected from the group consisting of radio, infrared, and blue-tooth transmission interfaces, as desired. In the present embodiment, a wireless transmission interface serves to provide signal connection for the present invention.

Referring to FIG. 3, the temperature sensing member 13 comprises a second interface unit 131 and a signal processing unit 133. The second interface unit 131 can be a wired transmission interface or a wireless transmission interface selected from the group consisting of radio, infrared, and blue-tooth transmission interfaces corresponding to the first interface unit. In the present embodiment, a wireless transmission interface is employed in the present invention. However, any transmission interface can be employed, as long as the first interface unit 113 corresponds to the second interface unit 131.

The temperature sensing member 13 serves to measure a body temperature of a test subject using a measuring unit (not shown) which can be integrated into the second interface unit 131 or the signal processing unit 133. Also, the measuring unit can be a structure made of temperature sensing materials or elements, or, alternatively, can be a chip having a function of sensing temperature (such as the one disclosed by Taiwan Patent No. 494459) which is combined with the second interface unit 131. The temperature sensing member 13, which can be made of soft materials, is attached onto the head or neck of the test subject, such that the body temperature of the test subject can be directly measured. Alternatively, the temperature sensing member 13 can be set on the test subject using a detachable fixing unit (not shown). The fixing unit can be selected from the group consisting of ventilated tapes, elastic covers, OK bands and bandages, or other elements which can be repeatedly attached on the human body.

In other words, the temperature sensing member 13 can be a structure which directly attaches on the test subject, or alternatively, can be directly attached onto the test subject by the foregoing detachable fixing unit, as long as the measuring unit comprised in the temperature sensing member 13 can directly contact the body of the test subject. In the present embodiment, the temperature sensing member 13 is attached onto the head or neck of the test subject; however, this placement is not intended to limit the scope of the invention. The temperature sensing member 13 can be attached or set at other locations on the test subject where measuring the body temperature is possible, as long as the body temperature of the test subject can be accurately measured.

The second interface unit 131 serves to transmit the result of measurement from the temperature sensing member 13 to the body 11 via the first interface unit 113. The signal processing unit 133 can be connected to or separated from the temperature sensing member 13, as long as the signal processing unit 133 is able to process and convert the result of measurement, and subsequently transmit the processed and converted result of measurement from the temperature sensing member 13 to the first interface unit 113 via the second interface unit 131. The signal processing unit 133 can be a microprocessor chip or any element having a similar function.

In other words, structures or functions of the second interface unit 131 and the signal processing unit 133 comprised in the temperature sensing member 13 can be modified depending on practical requirements, as long as the body temperature of the test subject can be accurately measured and the result of measurement can be converted and processed.

As the temperature sensing member 13 can directly and accurately measure the body temperature of the test subject, the drawback of a low accuracy in body temperature measurement caused in the portable temperature sensing device known in the prior-art can be eliminated.

The techniques for fabricating the first interface unit, the second interface unit, the chip having a function of automatically sensing temperature and the microprocessor chip, and the theory and actions of converting and processing the result of measurement are all known in the prior-art, and thus are not further described.

A determining unit (not shown) can be further provided in the temperature sensing member 13, such that the determining unit is able to determine whether the converted and processed result of measurement falls outside a temperature range. The determining unit sends out an indication if the result of measurement falls outside the temperature range, whereas the determining unit takes no action if the result of measurement falls within the temperature range. Further, an action indicating unit can be externally connected to the determining unit, such that the action indicating unit can serve to send out an indication. The action indicating unit serves to indicate whether or not the result of measurement falls outside upper and lower limits of the temperature range, and can be selected from the group consisting of an indicating lamp, a buzzer, a vibrator and/or other appropriate elements. The action indicating unit may also indicate additional operating status conditions. Thus, a steady or pulsating light and/or a warning sound or vibration occurs when the result of measurement falls outside the upper and lower limits of the temperature range. Alternatively, the determining unit can also be a structure having an indicating lamp, a buzzer, a vibrator and/or other appropriate elements provided therein. Furthermore, the determining unit can be used to display the power supply status of the temperature sensing member 13, and the element for supplying power for the temperature sensing member 13 can be any power source (such as a battery) known in the prior-art.

When the temperature sensing member 13 is provided in the body 11, the temperature sensing device 1 proposed in the present invention can also directly measure the body temperature of the test subject, therefore providing a function of a prior-art temperature sensing device. Furthermore, the temperature can be switched between the Celsius (° C.) and Fahrenheit (° F.) temperature scales by the body 11, such that the result of measurement can be accepted by users in different geographical locations. Additionally, a connecting component (not shown) can be used to connect the body 11 and the temperature sensing member 13. Thus, the temperature sensing member 13 can be removed from the test subject and connected to the body 11, such that the temperature sensing member 13 and the body 11 can be connected together for directly measuring the body temperature of the test subject. The connecting component can be selected from the group consisting of concave/convex fitting elements such as locking pawls, rabbets, flanges and retainers, thread fitting elements such as screws and nuts, or other appropriate elements such as magnetic blocks and Velcro strips, as long as the connecting component is able to connect the body 11 and the temperature sensing member 13. Also, the structure of the connecting component can be modified without departing from the spirit of the invention.

Moreover, the temperature sensing member 13 can be set on the test subject at a certain location, whereas the body 11 is placed in another place. When the temperature sensing member 13 is set on the test subject, the body temperature of the test subject can be immediately measured to obtain an initial temperature parameter value, and the temperature sensing member 13 is able to update the temperature parameter value or record the next temperature parameter value after a certain period of time, for example, 90 seconds.

In other words, the temperature sensing member 13 can be designed to re-measure the body temperature of the test subject after a certain period of time and update the initial temperature parameter value obtained from the prior measurement to the new temperature parameter value obtained after re-measurement. Alternatively, the temperature parameter value obtained after re-measurement can be recorded as another data measurement, such that body temperature changes of the test subject can be observed and compared. The time interval between body temperature measurements is not limited to any particular interval, and may even be variable. Thus, sequential measurements can be performed and the time interval can be modified depending on practical requirements.

Therefore, when the temperature sensing member 13 and the body 11 are separately located, the temperature sensing member 13 is used to measure the body temperature of the test subject. Subsequently, the signal processing unit 133 of the temperature sensing member 13 serves to transmit a temperature signal converted from the result of measurement to the body 11 within a certain range, such that the result of measurement can be immediately displayed by the displaying unit 111. Thus, the body temperature of the test subject can be monitored in real-time by the body 11. In the present embodiment, the body 11 and the temperature sensing member 13 can be separated from each other by a certain distance to perform real-time monitoring. The actual distance depends on the distance over which the signals can be sent and received between the element comprised in the body 11 and the temperature sensing member 13. In other words, the distance between the body 11 and the temperature sensing member 13 is not limited to a certain length and can be designed based on practical requirements.

The Second Embodiment

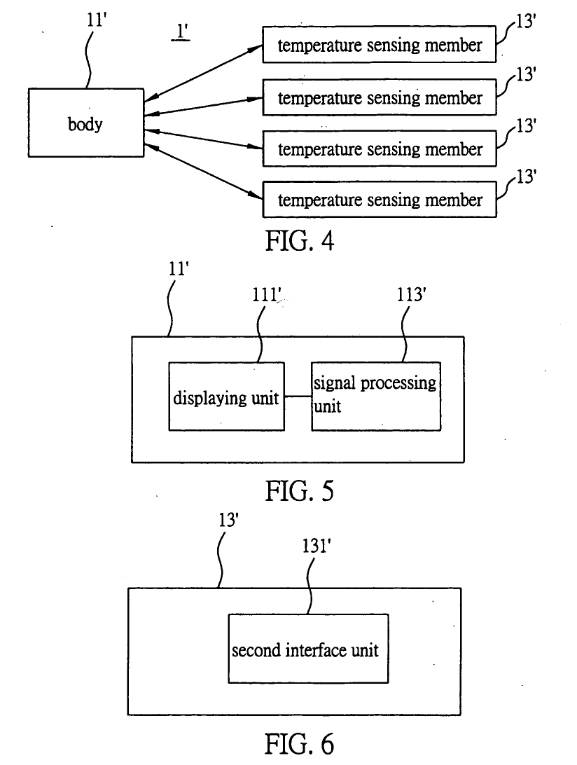

FIG. 4 to FIG. 6 are drawn according to the second embodiment of the present invention. A temperature sensing device 1′ comprises a body 11′ and a plurality of temperature sensing members 13′. The function of the temperature sensing device 1′ in the present embodiment is similar to that of the first embodiment. Therefore, only structures and functions different from the first embodiment are shown in describing the features and advantages for the present invention.

The present embodiment differs from the foregoing embodiment in that the temperature sensing device 1′ comprises a plurality of temperature sensing members 13′ which are respectively set on different test subjects, such that one body 11′ can be used to distantly and simultaneously measure and monitor body temperatures of these test subjects. Further, in order to minimize monitoring costs, a signal processing unit 113′ is provided in the body 11′ instead of in each of the temperature sensing members 13′. A first interface unit (not shown) is integrated into the signal processing unit 113′, and a second interface unit 131′ is integrated into each of the temperature sensing members 13′ for transmitting results of measurements from the temperature sensing members 13′ to the first interface unit of the signal processing unit 113′ of the body 11′. The received results are processed and converted to a temperature parameter value by the signal processing unit 113′, and the temperature parameter value is subsequently displayed by a displaying unit 111′ comprised in the body 11′. If desired, the temperature sensing member 13′ can be a combination of a chip having a function of sensing temperature and the function of providing the signal communications interface.

An action indicating unit (not shown) can be also provided in the body 11′. The action indicating unit may indicate the operational status of the system and serves to indicate whether any result of measurement falls outside the upper and lower limits of its corresponding temperature range. The action indicating unit can be selected from the group consisting of an indicating lamp, a buzzer, a vibrator and/or other appropriate elements. Thus, a steady of pulsed light and/or a warning sound or vibration can be produced when any result of measurement falls outside the upper and lower limits of its corresponding temperature range.

As the temperature sensing member 13′ is able to measure a temperature when the temperature sensing device 1′ proposed in the present invention is turned on, the body temperature of the test subject can be immediately obtained once the temperature sensing member 13′ has been attached to the test subject. Further, body temperature changes of the test subject can also be obtained after a certain period of time has elapsed.

For example, when parents need to monitor two children's body temperature changes, temperature sensing members 13′ can be attached to each child and the body 11′ can be placed in a central location, such that the parents are able to immediately monitor the both child's body temperature changes from one location. Additionally, when a company needs to monitor body temperature changes of employees (such as 50 employees), temperature sensing members 13′ can be attached to each of the employees, such that the company is able to monitor body temperature changes for all of the monitored employees from one central location.

When the temperature sensing device 1′ proposed in the present invention is used in places such as hospitals where body temperatures of multiple patients should be strictly monitored, temperature sensing members 13′ attached on each of the patients can remotely transmit body temperature changes for all the patients to the body 11′ located in a nursing center and immediately warn physicians and nurses if the body temperature of one or more patients falls outside the upper or lower limits of the established temperature ranges for the monitored patients. Therefore, not only are the body temperatures and body temperature changes of the patients monitored, but also the need for physicians and nurses to come into contact with patients can be reduced. In other words, body temperature changes of the patient can be accurately and continually reported to the attending physicians or nurses without unnecessarily jeopardizing the health of the physicians and nurses in the case of contagious patients.

Moreover, two or more bodies 11′ can be provided, such that a single observer is able to monitor body temperature changes of one or more test subjects from multiple locations. Alternately, multiple observers (such as the head of a department and a human resource manager) can monitor the body temperatures of one or more test subjects in a multiple-to-multiple manner. The number and locations of the body 11′ depends on the actual situation. Furthermore, when the temperature sensing device proposed in the present invention is used in different sections of a factory, different buildings of a hospital, different buildings of a community, or any other places where the body and the temperature sensing member are distantly separated from each other, a booster or other appropriate devices and elements can be used to ensure the connectivity of distance monitoring. Additionally, the distance of interface communication and strength of electromagnetic or optical waves generated can be adjusted depending on practical requirements. The adjustment technique is known in the prior-art and thus is not further described.

Therefore, the temperature sensing device 1′ in the present embodiment is able to obtain an accurate body temperature measurement without consuming a large amount of time in measuring the body temperature of the individual test subject and without affecting one's working efficiency while performing a distant body temperature measurement according to different real-time monitoring modes.

Accordingly, the temperature sensing device proposed in the present invention is capable of monitoring body temperature changes and improving the accuracy of measurement in that the body temperature of the test subject can be directly measured. Moreover, various economically-efficient modes, such as single-to-single, single-to-multiple, and multiple-to-multiple real-time temperature monitoring modes, can be performed to obtain an accurate record of the body temperature change of the test subjects.

It should be apparent to those skilled in the art that the above description is only illustrative of specific embodiments and examples of the present invention. The present invention should therefore cover various modifications and variations made to the herein-described structure and operations of the present invention, provided they fall within the scope of the present invention as defined in the following appended claims.

What is claimed is:

1. A temperature sensing device, comprising:
a body having a displaying unit and a signal processing unit; and
at least a temperature sensing member separated from the body by a predetermined distance, the temperature sensing member being provided with a detachable fixing unit, a measuring unit, and an interface unit, such that the temperature sensing member is set on a test subject by the fixing unit and body temperature of the test subject is measured by the measuring unit, wherein temperature measurements from the measuring unit are transmitted to the signal processing unit of the body by the interface unit, and subsequently converted and processed by the signal processing unit, such that the result of measurement being converted and processed is transmitted to and displayed on the displaying unit.

2. The temperature sensing device of claim 1, wherein the body further comprises an interface unit for communicating with the interface unit of the temperature sensing member.

3. The temperature sensing device of claim 1, wherein the body further comprises a determining unit for determining whether the measurements being converted and processed falls outside a temperature range, in which case an indication is sent out.

4. The temperature sensing device of claim 3, wherein the determining unit comprises an indicating lamp.

5. The temperature sensing device of claim 3, wherein the determining unit comprises a buzzer.

6. The temperature sensing device of claim 3, wherein the determining unit is externally connected to an action indicating unit for sending out an indication.

7. The temperature sensing device of claim 6, wherein the action indicating unit is selected from the group consisting of an indicating lamp, a buzzer, and a vibrator.

8. The temperature sensing device of claim 1, wherein the displaying unit is a screen.

9. The temperature sensing device of claim 1, wherein the fixing unit is selected from the group consisting of ventilated tapes, elastic covers, OK bands, and bandages.

10. The temperature sensing device of claim 1, wherein the measuring unit is a structure made of temperature sensing materials.

11. The temperature sensing device of claim 1, wherein the measuring unit is a chip having a function of automatically sensing a temperature.

12. The temperature sensing device of claim 1 or claim 11, wherein the temperature sensing member is a combination of the chip having a function of automatically sensing a temperature and the interface unit.

13. The temperature sensing device of claim 1, wherein the signal processing unit is a microprocessor chip.

14. The temperature sensing device of claim 1, further comprising a connecting component for removing the temperature sensing member from the test subject and connecting the temperature sensing member to the body, such that the temperature sensing member and the body can be combined together for directly measuring the body temperature of the test subject.

15. The temperature sensing device of claim 14, wherein the connecting component is selected from the group consisting of concave/convex fitting elements such as locking pawls, rabbets, flanges, and retainers, or thread fitting elements such as screws and nuts, or other appropriate elements such as magnetic blocks and Velcro strips.

16. A temperature sensing device, comprising:
a body having a displaying unit and a first interface unit; and
at least a temperature sensing member separated from the body by a predetermined distance and connected to the body by the first interface unit, the temperature sensing member being provided with a detachable fixing unit, a measuring unit, and a signal processing unit, such that the temperature sensing member is set on a test subject by the fixing unit and the body temperature of the test subject is measured by the measuring unit, where in measurements are converted and processed by the signal processing unit, and subsequently transmitted to the body via the first interface unit, such that the result of measurement is displayed by the displaying unit.

17. The temperature sensing device of claim 16, wherein the body further comprises a determining unit for determining whether the result of measurement being converted and processed falls outside a temperature range, in which case an indication is sent out.

18. The temperature sensing device of claim 17, wherein the determining unit comprises an indicating lamp.

19. The temperature sensing device of claim 17, wherein the determining unit comprises a buzzer.

20. The temperature sensing device of claim 17, wherein the determining unit is externally connected to an action indicating unit for sending out an indication.

21. The temperature sensing device of claim 20, wherein the action indicating unit is selected from the group consisting of an indicating lamp, a buzzer, and a vibrator.

22. The temperature sensing device of claim 16, wherein the fixing unit is selected from the group consisting of ventilated tapes, elastic covers, OK bands, and bandages.

23. The temperature sensing device of claim 16, wherein the measuring unit is a structure made of temperature sensing materials.

24. The temperature sensing device of claim 16, wherein the measuring unit is a chip having a function of automatically sensing a temperature.

25. The temperature sensing device of claim 16, wherein the measuring unit comprises a second interface unit.

26. The temperature sensing device of claim 16, wherein the signal processing unit is a microprocessor chip.

27. The temperature sensing device of claim 1, further comprising a connecting component for removing the temperature sensing member from the test subject and connecting the temperature sensing member to the body, such that the temperature sensing member and the body can be combined together for directly measuring the body temperature of the test subject.

28. The temperature sensing device of claim 27, wherein the connecting component is selected from the group consisting of concave/convex fitting elements such as locking pawls, rabbets, flanges, and retainers, or thread fitting elements such as screws and nuts, or other appropriate elements such as magnetic blocks and Velcro strips.
